(19) United States
(12) Patent Application Publication
(10) Pub. No.: US 2009/0304826 A1
(43) Pub. Date: Dec. 10, 2009

(54) Method and composition for dermatoses

(75) Inventor: Edward M. Lane, Weston, CT (US)

Correspondence Address: ROTHWELL, FIGG, ERNST & MANBECK, P.C., 1425 K STREET, N.W., SUITE 800, WASHINGTON, DC, 20005, US

(73) Assignee: Fairfield Clinical Trials, LLC, Bridgeport, CT (US)

(21) Appl. No.: 12/457,115

(22) Filed: Jun. 1, 2009

Related U.S. Application Data
(60) Provisional application No. 61/057,700, filed on May 30, 2008.
(60) Provisional application No. 61/088,440, filed on Aug. 13, 2008.
(60) Provisional application No. 61/118,191, filed on Nov. 26, 2008.
(60) Provisional application No. 61/159,984, filed on Mar. 13, 2009.

(51) Int. Cl. A61K 36/185; A61K 31/4545; A61K 31/445; A61K 31/495; A61K 31/55; A61P 17/02

(52) U.S. Cl. 424/725; 514/290; 514/317; 514/255.04; 514/217.05

(57) ABSTRACT

The invention provides a method and composition for treating dermatoses by topical application of a composition containing an antihistamine, an NSAID and optionally a botanical medicinal compound. The compositions are useful for skin conditions such as inflammatory skin conditions, including eczema, atopic dermatitis, non-allergic dermatitis, psoriasis and rosacea, or any inflammation of the skin.

### CROSS REFERENCE TO APPLICATIONS

This application claims benefit of U.S. Provisional Patent Application Ser. No. 61/057,700, filed May 30, 2008; Ser. No. 61/088,440, filed Aug. 13, 2008; Ser. No. 61/118,191, filed Nov. 26, 2008; and Ser. No. 61/159,984, filed Mar. 13, 2009. The disclosures of each of these provisional applications is incorporated by reference herein in its entirety.

### BACKGROUND OF THE INVENTION

1. Technical Field

This invention generally relates to the field of medical dermatology, allergy and cosmetics. This application describes and claims a topically applied medical treatment composition and methods, which can provide improvement in dermatoses, including, for example, eczema, atopic dermatitis, non-allergic dermatitis, psoriasis and rosacea, or any inflammation of the skin.

2. Description of the Background Art

Dermatitis generally refers to any inflammation of the skin, and may be caused by allergic reaction, irritants (such as poison ivy), drugs or infection by bacteria, viruses, parasites or fungi. Important types of dermatitis include allergic contact dermatitis, atopic dermatitis, eczema, psoriasis, and seborrhea. The term eczema often is applied to a range of skin conditions with symptoms including redness, skin edema (swelling), itching, flaking, etc. Eczema includes atopic eczema, contact dermatitis, xerotic eczema, seborrhoeic dermatitis, dyshidrosis, discoid eczema, venous eczema, neurodermatitis and autoeczematization, for example. Treatment for eczema has been aimed at controlling symptoms, since there is no known cure for eczema. Atopic dermatitis generally describes conditions where the skin reacts to irritants or allergens and becomes inflamed and very itchy. In most cases, treatment centers on preventing the condition by avoiding contact with substances or conditions known to trigger a reaction.

Psoriasis is an autoimmune condition which affects the skin, causing inflammation and excess skin production. The most common form is plaque psoriasis, a condition marked by scaly, inflamed areas on the skin and, usually, considerable itching. The cause of this condition is poorly understood, but may be related to stress, physical injury or infection. Treatment usually is based on trial-and-error.

Rosacea is diagnosed in approximately 13 million Americans. Although not a life threatening condition, it produces conspicuous facial redness and blemishes that can have a deep impact on a patient's self-esteem and quality of life. Rhinophyma, the most prominent feature of advanced rosacea, is often mistakenly associated with alcoholism, further stigmatizing Rosacea patients.

Rosacea is a chronic inflammatory process of the skin that typically affects the cheeks, nose, chin, forehead, eyes, and eyelids. The redness (erythema) of rosacea is caused by inflammation of the skin and dilation of the superficial vasculature, which is affected by various triggers including harsh soaps, sun damage, too aggressive skin treatments, alcohol, and acidic foods. Irritated skin areas develop blood vessel prominence and skin swelling (hyperplasia). Eighty-two percent of rosacea patients also suffer from acne and 35% have seborrheic dermatitis. Edema can develop as a result of the increased blood flow in the superficial vasculature. This edema might contribute to the last stage: fibroplasias and rhinophyma. The exact pathogenesis of rosacea is unknown.

The first step in the treatment of rosacea usually is avoidance of triggers that can cause a flare-up of the flushing or exacerbate the skin changes of rosacea. In addition, care must be taken to use only facial cleaners, lotions, and cosmetics that are non-irritating, hypoallergenic, and non-comedogenic. Rosacea should be treated at its earliest manifestations to mitigate the progression to the states of edema and irreversible fibrosis. Antibiotics have traditionally been considered the first line of therapy. Other therapies, including azelaic acid, topical retinoic acid, and topical Vitamin C have also been used, but with varying success. For recalcitrant rosacea, oral isotretinoin is often used, although it does have serious side effects, including teratogenic potential. Therefore, there is a need in the art for treatments for rosacea and other dermatoses, such as atopic dermatitis, eczema, or any skin inflammation. In particular, there is a need to find new and innovative ways to treat rosacea without use of oral medication, lasers, and harmful chemicals that could potentially harm the rosacea skin tissue.

### SUMMARY OF THE INVENTION

Therefore, embodiments of the invention relate to a topical composition which comprises an antihistamine compound, an anti-inflammatory drug compound and optionally contains a botanical medicinal agent. Preferably, the anti-inflammatory drug compound is a non-steroidal anti-inflammatory drug compound.

Preferred compositions contain about 0.0001% to about 99% of the antihistamine by weight, or about 0.0001% to about 50%, about 0.001% to about 10%, about 0.01% to about 5%, about 0.1% to about 3%, about 0.5% to about 2%, or about 1% of the antihistamine by weight.

Preferred compositions also contain about 0.01% to about 50% of the anti-inflammatory drug compound by weight, or about 0.0001% to about 99% of a non-steroidal anti-inflammatory drug (NSAID) compound by weight. Most preferred compositions contain about 0.0001% to about 50% of an NSAID compound by weight, or about 0.001% to about 10%, about 0.01% to about 5%, about 0.1% to about 3%, about 0.5% to about 2%, or about 1% of the NSAID compound by weight.

Preferred compositions contain a botanical medicinal agent selected from the group consisting of Achillea millefolium, Actaea racemosa, Allium sativum, Anethum graveolens, Amorphophallus konjac, Aquilare agollocha, Arnica spp., Aristolochia rotunda, Artemisia annua, Artemisia absinthium, Arum maculatum, Asimina spp., Astralagus membranaceus, Citrus aurantium, Crataegus spp., Cymbopogon spp., Cynara cardunculus, Digitalis lanata, Echinacea purpurea, Glycyrrhiza glabra, Hydratis canadensis, Hypericum perforatum, Lentinula edodes, Magnolia spp., Marrubium vulgare, Matricaria recruita, Melaleuca spp., Mentha piperita, Nepeta cataria, Nigella sativa, Oregano vulgare, Passiflora spp., Petasites spp., Phytolacca spp., Plantago spp., Poppiocious seediousphylla, Punica granatum, Rosa canina, Rubus fruticosis, Salvia lavandulaefolia, Salvia stenophylla, Sambocus spp., Serenoa repens, Silybum spp., Symphytum officinale, Tanacetum parthenium, Tanacetum officinale, Tilia spp., Urtica dioica, Vaccinium spp., Valeriana officinalis, Vanilla spp., Verbascum thapsus, and Zingiber officinalis. Most preferred compositions contain Calendula, preferably Calendula officinalis. In the preferred compositions, Calendula is present in an amount of about 0.0001% to about 99% Calendula by weight, or about 0.0001% to about 50%, about 0.001% to about 10%, about 0.01% to about 5%, about 0.1% to about 3%, or about 0.5% to about 2% Calendula by weight.

In preferred compositions, the antihistamine is selected from the group consisting of fexofenadine, loratadine, desloratadine, azelastine, cetirizine and levocetirizine and the anti-inflammatory compound is ibuprofen.

Embodiments of the invention also relate to methods of treating dermatoses in a human in need thereof, which comprises topically applying to the skin of said human a topical composition as described above and to methods of treating swelling, redness, darkness or inflammation of the skin of a human in need thereof, which comprises topically applying to said skin a topical composition as described above. In some embodiments, the methods are for treating rosacea in a human in need thereof, in any skin area, but preferably on the skin of the face or the eye area. Such methods can involve applying about 0.0001 cc to about 1 cc of said topical composition per 1-2 or 1-10 square inch skin area, or about 0.001 cc to about 0.5 cc, about 0.01 cc to about 0.3 cc or about 0.1 cc to about 0.2 cc to the same skin area.

### DETAILED DESCRIPTION OF THE PREFERRED EMBODIMENTS

Histamine, (2-(4-imidazolyl)-ethyl-amine; 4-aminoethylglyoxaline), is a dibasic vasoactive amine that is located in most body tissues, but is highly concentrated in the lungs, skin, and gastrointestinal tract. Histamine is stored in mast cells and basophils. Ionic forces within intracellular granules hold histamine by macroheparin. The interaction between allergen and IgE, bound to the surface of mast cells and basophils by a surface receptor that binds the Fc fragment of IgE, leads to degranulation of these cells, with release of mediators, such as histamine, leukotrienes, substance P, interleukins, bradykinins and kallikreins, among others. Histamine's main physiological actions include stimulation of gastric secretion, contraction of most smooth muscle, cardiac stimulation, vasodilatation, and increased vascular permeability.

Acting on H1 receptors, histamine causes the dilation of blood vessels. It induces endothelial cells to synthesize vascular smooth muscle relaxants, including prostaglandin and nitric oxide, which cause vasodilatation. Vasodilatation of small arterioles and precapillary sphincters causes reddening, while increased permeability of post capillary veins causes the wheal. Histamine also induces the release of a vasodilating mediator, thus producing the flare. Histamine produces many of the effects of inflammation and hypersensitivity, including vasodilatation, edema, increased vascular permeability, and smooth muscle contraction. Increased vascular permeability causes fluid to escape from capillaries into the tissues, which leads to the classic symptoms of an allergic reaction: a runny nose and watery eyes. It is thought to be the major mediator of the acute inflammatory response, although histamine H1 antagonists have little effect on acute inflammation. Histamine produces many of the effects of inflammation and hypersensitivity, including vasodilation, edema, increased vascular permeability, and smooth muscle contraction.

Antihistamines are indicated, Federal Drug Administration (FDA) approved, and commonly used for treatment of allergies, motion sickness, certain types of headaches, Crohn's disease, acute flare-ups of multiple sclerosis, and some stomach secretory conditions. Although widespread in use, major central nervous system adverse effects such as sedation and performance deficits, and their anticholinergic activities have introduced problems with their widespread usefulness. Antihistamines are a broad class of pharmacologic agents that include first generation, centrally acting H1-receptor antagonists (such as diphenhydramine) and the newer, second generation, non-sedating H1 blockers (e.g. fexofenadine, loratidine, desloratidine, azelastine, cetirizine, and levocetirizine). Other antihistaminic agents, such as cimetidine, work primarily at H2 receptors causing inhibition of gastric secretion. Other experimental antihistamines act on presynaptic H3 and H4 receptors.

H1 histamine antagonists are reversible inhibitors of histamine receptors. First generation H1-receptor blockers are also potent competitive inhibitors of muscarinic receptors and may cause anticholinergic syndrome (e.g. sinus tachycardia, dry skin, dry mucous membranes, dilated pupils, ileus, urinary retention, and agitated delirium). Second generation antihistamines, such as fexofenadine, loratidine, desloratidine, azelastine, cetirizine, and levocetirizine, among others, are peripherally selective H1 receptor antagonists. They have a distinct advantage over previous compounds because they bind much more selectively to peripheral H1 receptors and have a lower binding affinity for the cholinergic and alpha-adrenergic receptor sites than first generation antihistamines. Second generation antihistamines therefore have less anticholinergic and alpha-adrenergic effect than first generation antihistamines, and cause less vasodilatation and capillary permeability. In addition, they are lipophobic and therefore do not pass easily across the blood-brain barrier, thus causing much less sedation. These second generation antihistamines are popular for treatment of allergic reactions because their specificity for the peripheral histamine receptor site reduces or eliminates many adverse side effects. Any H1, H2, H3, H4, or any combination thereof is contemplated for use in this invention, although H1 histamine antagonists or blockers (antihistamines) are preferred. Antihistamines do improve itchiness but do not improve skin inflammation associated with dermatitis when taken systemically (orally) or when used alone topically.

Ibuprofen, 2-[4-(2-methylpropyl)phenyl] propanoic acid, is a non-selective non-steroidal anti-inflammatory drug (NSAID) that also exhibits analgesic and antipyretic properties. Ibuprofen is used orally for anti-inflammatory and analgesic effects in the symptomatic treatment of mild to moderate pain or fever. The majority of NSAID compounds, including ibuprofen, are believed to work through inhibition of cyclo-oxygenase (COX), thus inhibiting prostaglandin synthesis. Ibuprofen inhibits both cyclo-oxygenase-1 (COX-1) and cyclo-oxygenase-2 (COX-2), although it is believed that ibuprofen's analgesic, antipyretic, and anti-inflammatory activities are achieved principally through COX-2 inhibition. COX-1 inhibition is believed to be responsible for Ibuprofen's unwanted side effects on platelet aggregation and on the gastro-intestinal mucosa (e.g. gastritis, ulceration, and/or bleeding). Any NSAID or other compound that inhibits COX-1, COX-2, COX-3 or any combination thereof, and in general anti-inflammatory compounds, are contemplated for use as part of this invention, although non-specific NSAID compounds and COX-2 inhibitors generally are preferred.

NSAID compounds that are contemplated for use with the invention include salicylates, arylalkanoic acid NSAIDS, arylpropionic acid NSAIDS, N-acylanthranilic (fenamic) acid NSAIDs, pyrazolidine derivative NSAIDS, oxicam NSAID compounds, selective COX-2 inhibitors, sulphonamilide NSAID compounds, and other compounds such as 5-LOX/COX inhibitors. The term NSAID, as used herein, therefore refers to any non-steroidal anti-inflammatory drug or COX (COX-1, COX-2 or COX-3) inhibitor compound. Non-limiting examples of suitable compounds are acetylsalicylic acid (aspirin), ampyrone, celecoxib, diclofenac, diflunisal, droxcam, ibuprofen, indomethacin, licofelone, mefanamic acid, naproxen, nimesulide, omega-3 fatty acids, phenylbutazone, proicam, rofecoxib, valdecoxib or any combination thereof.

The botanical medicinal agent (herbal) component of the inventive composition can include a whole herb preparation or an extract of some form. Suitable extracts include, but are not limited to tinctures (alcohol extract), elixirs (alcohol extract), tisanes (hot water extract), decoctions (long-term boiled extracts), macerates (cold infusions), vinegars, infusions, essential oils (oil extract), salves, oils, balms, creams, lotions or syrups. Extracts such as those listed above may be dried to form a powder and may be prepared for administration as a liquid, solid or semi-solid/semi-liquid preparation, or any topical preparation such as a cream or lotion. The extracts can be diluted in a liquid (aqueous or oil) carrier or a solid (powder or other) carrier. Whole herb preparations can be dried whole herbs, fresh herbs or juice, for example. Such whole herb and extract preparations can be administered orally, by inhalation, or, (preferably for the present invention) topically.

Calendula officinalis (pot marigold) is a member of the Compositae family and is the preferred botanical medicinal agent. Preferred preparations of this plant are infusions, tinctures, or liquid extracts of the plant flowers, formulated into a topical preparation such as a cream or lotion. However, any of the botanical medicinal agent (herbal) compounds of Table 1, below, are contemplated for use with the invention.

The inventors here have discovered that a combination of an antihistamine, an anti-inflammatory drug compound and a botanical medicinal agent such as those listed in Table 1, below, preferably Calendula (Calendula officinalis or other species) is surprisingly effective in treating rosacea. When used in combination, the decreased alpha-adrenergic effect of second generation antihistamines lessens the rise in blood pressure caused by ibuprofen, resulting in less blood flow to the area and less venous engorgement. Without wishing to be bound by theory, these factors are believed to result in a superior effect on swelling and skin inflammation in general. The preferred inventive products therefore combine an antihistamine, preferably a second generation antihistamine, together with an NSAID, preferably ibuprofen. Other embodiments include an antihistamine with an anti-inflammatory drug and optionally a botanical medicinal agent.

Clinical trials showed that although treatment with a 1% Calendula product topically had no efficacy alone, addition of 1% Calendula to a composition also containing 1% fexofenadine and 1% ibuprofen resulted in a significant improvement in results for improvement of rosacea over the fexofenadine/ibuprofen composition alone. See Example 5.

Antihistamine compounds that are useful in the inventive compositions include any H1 receptor-inhibiting compound, therefore, the term “antihistamine” as used herein, refers to any such compound. Specific examples of the “first generation” H1 antihistamines include but are not limited to acrivastine, brompheniramine, chlorpheniramine, clemastine, diphenhydramine, doxylamine, hydroxyzine, pheniramine, and promethazine. Specific examples of the preferred “second generation” H1 antihistamines include, but are not limited to azelastine, cetirizine, desloratidine, fexofenadine, levocetirizine, loratidine, and olopatadine. Useful compounds may be members of any of the 7 structural classes of antihistamine (alkylamines, ethanolamines, ethylenediamines, phenothiazine, piperidines, piperazines or norpiperidine, imidazoazepines). Other compounds which may be used in the inventive compositions as additional optional active agents are vitamin K, vitamin C, grape seed oil, caffeine, pseudoephedrine, ephedrine, topical bleaching agents, steroids, alkanolamines, Hylexin®, retinol, and tetrapeptides. Preferred antihistamine compounds are fexofenadine, loratadine, desloratadine, azelastine, cetirizine and levocetirizine.

In addition to non-steroidal anti-inflammatory compounds, steroid anti-inflammatory compounds and/or leukotriene blockers also are contemplated as part of this invention and are included in the term “anti-inflammatory drug compound.” These compounds may be used instead of an NSAID compound or in addition to an NSAID compound. For example, steroids (including glucocorticoids, mineral corticoids and adrenal-corticoids) include, but are not limited to, cortisone, hydrocortisone, prednisone, prednisolone, triamcinolone, beclomethasone, ciclesonide, methylprednisolone, betamethasone, fludrocortisone, DOCA, aldosterone, estrogen, androgen, and progesterone. Suitable leukotriene blockers include but are not limited to monoleukast, zileuton and zafirlukast.

Appropriate concentrations of the antihistamine compound for the inventive compositions range from about 0.0001% to about 99% or about 0.0001% to about 50% antihistamine compound by weight in a suitable vehicle. Preferably, the compositions contain about 0.001% to about 10% antihistamine or about 0.01% to about 5% antihistamine by weight. Most preferred compositions contain about 0.1% to about 3% or about 0.5% to about 2%, or about 1% antihistamine compound by weight. These percentage concentrations are approximately equivalent to 0.0001 mg antihistamine per cc of the composition to about 25 mg antihistamine per cc of the composition or about 0.001 to about 10, about 0.01 to about 5, about 0.1 to about 3, about 0.5 to about 2, or about 1 mg antihistamine per cc of the composition.

Inventive compositions also contain an anti-inflammatory compound. If the anti-inflammatory compound is an NSAID, such NSAIDs preferably are present in concentrations in the range from about 0.0001% to about 99% NSAID compound or about 0.0001% to about 50% NSAID by weight in a suitable vehicle. Preferably, the compositions contain about 0.001% to about 10% NSAID or about 0.01% to about 5% NSAID by weight. Most preferred compositions contain about 0.1% to about 3% or about 0.5% to about 2%, or about 1% NSAID compound by weight.

The concentrations of the combined active agents (antihistamine and NSAID) therefore can range from 0.0002% to substantially 100%, although the preferable concentration is 1-10%, 1-5%, or 1-2%. Each active agent preferably is present at 1-2%. Useful percentage concentrations are approximately equivalent to 0.0001 mg NSAID per cc of the composition to about 25 mg NSAID per cc of the composition or about 0.001 to about 10, about 0.01 to about 5, about 0.1 to about 3, about 0.5 to about 2, or about 1 mg NSAID per cc of the composition. Desirable concentrations of steroid compounds are about 0.01% to about 50% by weight and preferably about 1% to about 10%, to provide a dosage per use of about 0.0001 mg to about 10 mg and preferably about 0.01 mg to about 0.1 mg of the steroid compound. These same ranges of concentration are suitable for leukotriene blocker compounds as well.

In addition to the primary botanical medicinal agent, such as the botanicals listed in Table 1, below, the inventive compositions also may contain other botanical medicinal ingredients and extracts to reduce inflammation, redness, swelling, and to promote healing. Suitable botanical medicinal agents include most preferably Calendula, such as Calendula officinalis and the like. Other botanical medicinal agents include sea weed, algae, plankton, and plant stem cells, or an extract thereof. The botanicals or extracts thereof preferably are present in concentrations in the range from about 0.0001% to about 99% or about 0.0001% to about 50% by weight in a suitable vehicle. Preferably, the compositions contain about 0.001% to about 10% or about 0.01% to about 5% by weight. Most preferred compositions contain about 0.1% to about 3% or about 0.5% to about 2%, or about 1% botanical compound by weight.

The inventive compositions also may optionally contain additional ingredients that can promote soothing of the skin or health of the skin, including ingredients that promote a youthful appearance. Such optional ingredients can include botanical extracts such as aloe or green tea, vitamins and antioxidants such as vitamin C, vitamin A or retinol, vitamin E, vitamin K, astringents, hyaluronic acid, omega-3 fatty acids, sunscreen, grape seed oil, caffeine, pseudoephedrine, ephedrine, topical bleaching agents, alkanolamines, Hylexin® and/or tetrapeptides. When the anti-inflammatory compound is an NSAID, the compositions also can include an additional anti-inflammatory compound such as a steroid and/or a leukotriene blocker. Therefore, the term “anti-inflammatory drug compound” includes combinations and mixtures of different anti-inflammatory drugs. Additionally, ingredients that aid in penetration of the active ingredients into and/or through the skin also optionally may be included in the inventive compositions.

The vehicle or carrier for the antihistamine and NSAID or other anti-inflammatory compound active compounds with a botanical medicinal agent may be any excipient or combination of excipients which are suitable for topical delivery of the active compounds to the skin of the face or body wherever irritation, redness, inflammation or any dermatitis or dermatosis has occurred, and particularly to the skin of the face. Preferred vehicles therefore include vegetable or mineral oils, lotions, creams, milks, gels, aqueous liquids, emulsions, ointments, liniments, unguents, rubs, balms, salves, sera, mists, powders, liposomes and any other suitable topical formulation.

Preferred vehicles are water-soluble or emulsions, have been clinically tested and do not cause eye or skin irritation. These vehicles and compositions made using them may be designed for application using the fingers or a suitable wand or swab, or may be provided in a container for application with a brush, wipe, swab, roller, spray, pen, pump or the like to deliver a precise or approximate amount of the product. The compositions may be applied in any convenient manner as discussed above. Most commonly, the composition is formulated in a liquid, semi-liquid, or gel formulation. In such cases, a convenient amount of the composition is applied to the skin, for example the eye area, cheeks, nose and/or chin area of the face and gently blended into the skin with the fourth (ring) finger. Alternatively, the composition may be applied using an applicator device and optionally massaged into the skin. The inventive compositions also may contain inert ingredients such as dyes and colorants, cosmetic tints or pigments to temporarily conceal discolored or red and inflamed areas, fragrances or flavorings, humectants, emollients, emulsifiers and surfactants, pH modifiers, binders, thickeners, and/or preservatives.

Although a primary use of the inventive composition is to treat rosacea and other dermatoses such as eczema and psoriasis, the inventive compositions can be used on any area of the skin, wherever an anti-inflammatory action is desired. For example, the compositions can be used on bruises, insect bites, allergic wheals, sunburn and the like, or wherever inflammation of the skin has occurred, such as in eczema, atopic dermatitis, psoriasis, poison ivy, poison oak, or poison sumac. In addition, the compositions can be applied to areas of the skin after cosmetic procedures such as laser treatments, electrolysis, waxing, peels (such as chemical peels) injections, piercings or tattoos to reduce swelling and inflammation and speed recovery.

Preferred methods of using the product involve application to the skin of an amount of the composition of about 0.0001 cc to about 1 cc to an area of about 1-10 square inches, for example to the face or any affected area of the skin, at least 1 or 2 times per month and up to 6 times per day. Any dosage schedule, such as once-a-week, once-a-day, or periodic use when the need arises is contemplated and within the scope of this invention. Preferably, the compositions described herein are applied to the skin about 2 times per day, using about 0.01 to about 1 cc or most preferably about 0.1 to about 0.2 ccs for each square inch of skin surface. Because the topical preparations of this invention are safe to use and non-irritating, the precise amount used is not critical. Therefore, dosage schemes and schedules outside these suggested ranges are contemplated for use.

In a study designed to test the effectiveness of the inventive compositions for amelioration of eye area darkness and swelling, a composition containing 1% fexofenadine and 1% Ibuprofen, dissolved in a water-based inert vehicle, was applied topically to the area under the eyes of 59 patients. This treatment resulted in a statistically significant improvement in appearance in all patients compared with the vehicle alone. Furthermore, in other studies, the product containing 1% fexofenadine and 1% ibuprofen was more effective in reducing dark circles and lower eye area swelling than either drug alone when applied topically. A third study found that application of an inventive composition (1% fexofenadine and 1% ibuprofen) topically resulted in statistically significant improvements in the appearance of dark circles and lower eye area swelling compared to systemic (oral) delivery of the two drugs in combination. Therefore, the inventive compositions have been shown to be more effective than comparable compositions delivered orally and more effective than either active component alone applied topically for swelling of the skin.

All references cited in the specification are hereby incorporated by reference in their entirety.

### EXAMPLES

### Example 1

### Double-Blind Placebo-Controlled Human Study of Topical Composition

An embodiment of the inventive composition, referred to as the “test article” in this example, contained 1% 2-[4-(2-methylpropyl)phenyl] propanoic acid and fexofenadine in a vehicle of Kiehl's® brand Eye Alert®, a commercially available eye cream. The compositions were prepared aseptically. The test article and vehicle alone were placed in numbered, blinded syringes, with a safety cap in place.

In a double-blinded, placebo-controlled study in humans, 59 male and female patients, 18 years of age and older, received either test article (inventive composition) or vehicle alone (control) to apply topically to the under-eye area of the skin. Each patient applied the test article to one side and the vehicle to the other, using a clean cotton-tipped applicator for each application per side, so that each patient could serve as her own control. The patients received two syringes, one with vehicle (control) and one containing the test article, with instructions about technique to apply the creams two times a day. Pre-treatment photos were obtained.

After one and two weeks, all patients completed a self-assessment. A blinded examiner then rated the patients in terms of clinical improvement and differences between the two sides. The amount of test article remaining was weighed and returned at the end of Week 1 and weighed and collected at the end of Week 2. The patients returned again after one week off-therapy (the end of Week 3) to be photographed and to complete a self-assessment. The same blinded examiner scored the patients in terms of clinical results.

Patients were treated for 4 weeks, twice a day (through Visits 1-5) with active drug to one side of the face and placebo (vehicle alone) to the other side of the face. The patient and the investigator were blinded as to which side was treated with which formulation. At weekly visits on therapy (Visits 2-5) and two weeks off therapy (Visit 6), both the patients themselves and the Investigator rated the patients in terms of clinical improvement or worsening and differences between the two sides. Patients were photographed at each visit. The test articles were weighed to assure compliance.

All patients reported a significant improvement in lower eye area swelling on the test article side versus the vehicle alone side. The examiner agreed with these assessments. There were no adverse events.

### Example 2

### Effectiveness of Oral Combination Therapy Compared to Topical Combination Therapy in Humans

After informed consents were obtained, seven women, age 18 or older, received oral medication, 100 mg Ibuprofen and 60 mg Fexofenadine, to be taken twice daily for one week. The medications were delivered in a blister pack to lessen the chance of dosing error and to increase compliance. The blister packs for the oral medication were returned at the end of Week 1. At that time, the women received a topical treatment composition containing 1% fexofenadine and 1% ibuprofen, dissolved in a stable and commercially available eye cream, to be applied to each side under the eyes, twice a day, using a clean cotton tipped applicator. Each applicator was to be used once only and for only one side. The patients returned the test article at the end of Week 2 for weighing of the test article. Photographs of the peri-orbital areas of all patients were taken at entry, end of Week 1, and end of Week 2. The oral combination fexofenadine and ibuprofen caused no improvement in the lower eye area swelling, but there was a significant improvement in the appearance of swelling in all patients as early as Day 3 after beginning combination topical therapy.

### Example 3

### Effectiveness of Topical Single-Drug Therapy Compared to Topical Combination Therapy in Humans

After informed consent was obtained, six female patients, age 18 and older, received a composition according to an embodiment of the invention containing 1% fexofenadine and 1% ibuprofen in an inert eye cream and control compositions containing vehicle plus 1% fexofenadine or vehicle plus 1% ibuprofen. The patients were instructed to apply the products with a clean cotton tipped applicator topically to the under-eye area, each on one side of the face with a separate clean cotton tipped applicator.

During Week 1, the patients applied the combination product to one side and either fexofenadine alone or ibuprofen alone (chosen at random for the week) to the other side. During Week 2, treatment with the combination product was maintained on the same side of the face, while the other side was switched from one compound alone to the other (i.e., from fexofenadine to ibuprofen or from ibuprofen to fexofenadine). Photographs and patient satisfaction surveys were administered at entry, at the end of Week 1, and at the end of Week 2. Topical antihistamine alone and topical ibuprofen alone did not improve lower eye swelling, but the combination 1% fexofenadine and 1% ibuprofen according to an embodiment of the invention caused a significant improvement in lower eye swelling.

### Example 4

### Topical Rosacea Treatment

Patients applied the inventive composition to be tested to the right side of the middle one third of the face and vehicle only to the left side of the middle one third of the face. Each patient therefore served as his/her own control, using a protocol testing the combination product (1% fexofenadine and 1% ibuprofen in Emollient Base Cream) versus vehicle (placebo) alone (Emollient Base Cream). The combination product was a sterile, multi-dose, preserved, combination eye cream product intended for topical, dermatologic use containing 1% ibuprofen and 1% fexofenadine in Emollient Base Cream. The Emollient Base Cream (“Vehicle”) was a commercially available Emollient Base Cream. Ingredients included: anhydrous ointment base, polysorbate 80, water, butylated hydroxytoluene, and sodium benzoate. Mixing and packaging were carried out under aseptic conditions and sterility of the final composition was assured by random checks by culture techniques. Composition verification and ninety (90) day shelf life stability were confirmed.

Each patient received the combination composition (1% fexofenadine and 1% ibuprofen in Vehicle) to be used on the rosacea on the left middle of the face. All compositions were supplied in 10 mL plastic, sterile syringes, sealed with a replaceable Luer lock stopper, and were stored in room temperature. Each container was color-coded and labeled with a randomized and unique patient number as well as instructions for use and space to write in the patient's name, number, and side of use. Patients were instructed to use the combination product on rosacea on the right side of the middle one third of the face and vehicle alone on rosacea on the left side of the middle one third of the face.

The compositions (0.1 cc) were applied with a clean, cotton tipped applicator to each side of the face, twice daily (morning and evening), for twenty-eight (28) days. Patients received written dosing instructions, and the dosing instructions were written on the composition containers. Confirmation of dosing was recorded using a phone-in diary system. The study coordinator demonstrated proper dosing techniques on a mannequin during the first (baseline) visit. The patient was observed by the study coordinator to self-administer the first dose. The patient then was responsible for self-administration of all remaining doses.

The clinical staff kept a current record of the inventory and dispensing of all compositions. All tested compositions were weighed and weights were recorded at each study visit, and at the return of the material at the end of the study. Any significant discrepancy and/or deficiency was recorded, with an explanation. All compositions were accounted for, and in no case were any compositions used in any unauthorized situation. After all patients had completed the study, a complete inventory of the composition was carried out.

Twenty (20) adult patients (age 18 and older), male and female, were enrolled. Discontinued patients were not replaced for any reason. Patients had rosacea on both sides of the face at entry. Other inclusion criteria were (1) ability to read, understand, sign, and comply with the terms of the informed consent; (2) agreement to comply with the requirements of the study; (3) willingness to be photographed and to possibly have those photographs disseminated; (4) absence of current bacterial or viral conjunctivitis or other condition which could interfere with the evaluation of the study drug; (5) negative pregnancy test and use of contraception for all menarcheal females; (6) no breast-feeding; (7) no history of active herpes simplex, herpes zoster, vaccinia, or varicella infections or overt viral infections of the eyes and/or surrounding structures (including nose and paranasal sinuses); (8) no prior ophthalmologic and/or facial cosmetic surgery within one year of study entry; (9) no concurrent immunosuppressive therapy (e.g. cancer chemotherapy) or known acute or chronic impaired renal function, renal failure, or hepatitis; (10) absence of insulin dependent diabetes; (11) no systemic disease or disorder, complicating factor, or structural abnormality that would negatively affect the conduct or outcome of the study (e.g. lupus, anticoagulation, platelet abnormalities, etc.); (12) no currently known or suspected infection requiring systemic antimicrobial therapy; (13) no bleeding disorders, cardiovascular disease, peptic ulceration, or a history thereof; (14) no use of prohibited medications; (15) no concomitant use of topical or oral analgesics (i.e. NSAID's, narcotics, and/or aspirin products) which may have anti-inflammatory properties; (16) no concomitant use of antihistamines (oral, topical or nasal) or use of other allergy or combination allergy medication; (17) no known or suspected allergy to ibuprofen, fexofenadine, or any of the components in Vehicle; (18) no therapy with another investigational drug, study medication, or device within the thirty (30) days prior to the Day 1 visit; (19) no patient from the same family or household enrolled into the study at the same time; (20) no family or household members of site personnel directly involved in the study; (21) no anticipated change in the use of any medication during the study that might affect the conduct or outcome of the study; and (22) any other sound medical reason. Patients were enrolled and evaluated at 6 visits:

(1) Baseline Visit 1, Day 1

(2) On therapy, Day 8

(3) On therapy, Day 15

(4) On therapy, Day 22

(5) End of Therapy, Day 29

(6) Test of Cure (TOC), day 43.

In addition to patient visits, the patients were required to make a daily phone call using a phone-in diary system in order to review the diary and dosing compliance.

At the beginning of the study, a complete clinical assessment was performed. The patient's rosacea was characterized according to the (1) discoloration of skin (type and degree); (2) skin eruption and integrity; and (3) degree of swelling of the skin. In addition, the rosacea was rated as to severity on a 1-10 scale and the patient's face was photographed with attention to the peri-orbital area. During the “on therapy,” “end of therapy” and “test of cure” visits, the patient's appearance was assessed by the same investigator and characterized according to (1) clinical response on a 4 point scale (resolved/cured; improved; not changed; worsened); (2) appearance of reaction to medication; and (3) change in skin tone and/or structure.

Twenty (20) adult patients, ages 33-67, mean age 46, fifteen (15) female and five (5) males, were enrolled in the study. Eighteen (18) were Caucasian and two (2) were Hispanic. Thirteen (13) patients were non-allergenic while five (5) had seasonal allergic rhinitis and two (2) had both seasonal and perennial rhinitis.

Of the 20 patients enrolled, all 20 completed the study. Of these 20 patients, 19 showed a significant improvement in the appearance of redness (erythema) on the treated side at Day 8 (p<0.001). The improvements continued throughout the treatment regimen (Day 29). Most patients (16 out of 20 (80%)) maintained their improvements off-therapy for two weeks (Day 43).

Patients rated their own appearance on a 1-6 scale, with 1 representing a sense of disfigurement and 6 representing normal appearance without any redness (erythema), pustules, papules, or dilated blood vessels. The averages on the treated side were:

| | |
|---|---|
| Visit 1 | 2.4 |
| Visit 2 | 4.8 |
| Visit 3 | 5.1 |
| Visit 4 | 5.0 |
| Visit 5 | 5.0 |
| Visit 6 | 4.6 |

The averages on the placebo side were essentially unchanged from Visit 1, with no worsening in appearance throughout. There were no adverse events and side effects reported by any of the patients.

A safe topical combination therapy, utilizing fexofenadine 1% and ibuprofen 1% in an Emollient Base Cream has been shown to dramatically, significantly, rapidly, and nearly universally improve the redness (erythema) and dilated blood vessels of rosacea.

### Example 5

### Topical Rosacea Treatment

Twenty patients with rosacea of the face were treated with either vehicle alone, 1% Calendula in vehicle, 1% fexofenadine and 1% ibuprofen in vehicle, or 1% Calendula, 1% fexofenadine, and 1% ibuprofen in vehicle, twice a day for four weeks. Patients were then followed for two weeks off-therapy. Results showed that the composition containing 1% Calendula, 1% fexofenadine, and 1% ibuprofen in vehicle provided significantly better results than the composition containing only 1% ibuprofen and 1% fexofenadine. Therefore, unexpectedly, while the 1% Calendula alone had no efficacy in improving the redness, erythema, inflammation, or dilated blood vessels of rosacea, its addition to the other components dramatically improved the effectiveness of the composition.

TABLE 1
Exemplary Botanical Medicinal Agents.

| Species | Common Name |
|---|---|
| Achillea millefolium | Yarrow |
| Actaea racemosa | Black Cohosh |
| Allium sativum | Garlic |
| Anethum graveolens | Dill |
| Amorphophallus konjac | Konjac |
| Aquilare agollocha | Eaglewood |
| Arnica spp. | Arnica |
| Aristolochia rotunda | Smearwort |
| Artemisia annua | Sweet Sagewort |
| Artemisia absinthium | Wormwood |
| Arum maculatum | Lords and Ladies |
| Asimina spp. | Pawpaw |
| Astralagus membranaceus | Astralagus |
| Citrus aurantium | Bergamot orange |
| Crataegus spp. | Hawthorn |
| Cymbopogon spp. | Lemon Grass |
| Cynara cardunculus | Artichoke |
| Digitalis lanata | Digitalis |
| Echinacea purpurea | Echinacea |
| Glycyrrhiza glabra | Licorice |
| Hydratis canadensis | Goldenseal |
| Hypericum perforatum | St. John's Wort |
| Lentinula edodes | Shiitake |
| Magnolia spp. | Magnolia |
| Marrubium vulgare | Horsehound |
| Matricaria recruita | Chamomile |
| Melaleuca spp. | Tea tree oil |
| Mentha piperita | Peppermint |
| Nepeta cataria | Catnip |
| Nigella sativa | Black Cumin |
| Oregano vulgare | Oregano |
| Passiflora spp. | Passion-flower |
| Petasites spp. | Butterbur |
| Phytolacca spp. | Pokeweed |
| Plantago spp. | Plantain/Psyllium |
| Poppiocious seediousphylla | Poppy seeds |
| Punica granatum | Pomegranate |
| Rosa canina | Rose hips |
| Rubus fruticosis | Blackberry |
| Salvia lavandulaefolia | Salvia |
| Salvia stenophylla | Blue Mountain Sage |
| Sambocus spp. | Elderberry |
| Serenoa repens | Saw Palmetto |
| Silybum spp. | Milk Thistle |
| Symphytum officinale | Comfrey |
| Tanacetum parthenium | Feverfew |
| Taracetum officinale | Dandelion |
| Tilia spp. | Lime Blossom |
| Urtica dioica | Stinging Nettle |
| Vaccinium spp. | Cranberry |
| Valeriana officinalis | Valerian Root |
| Vanilla spp. | Vanilla |
| Verbascum thapsus | Mullein |
| Zingiber officinalis | Ginger |

1. A topical composition which comprises an antihistamine compound, an anti-inflammatory drug compound and optionally comprises a botanical medicinal agent.

2. The composition of claim 1, wherein said anti-inflammatory drug compound is a non-steroidal anti-inflammatory drug compound.

3. The composition of claim 1, which contains about 0.0001% to about 99% of said antihistamine by weight.

4. The composition of claim 3, which contains about 0.0001% to about 50% of said antihistamine by weight.

5. The composition of claim 3, which contains about 0.001% to about 10% of said antihistamine by weight.

6. The composition of claim 3, which contains about 0.01% to about 5% of said antihistamine by weight.

7. The composition of claim 3, which contains about 0.1% to about 3% of said antihistamine by weight.

8. The composition of claim 3, which contains about 0.5% to about 2% of said antihistamine by weight.

9. The composition of claim 3, which contains about 1% of said antihistamine by weight.

10. The composition of claim 1, which contains about 0.01% to about 50% of said anti-inflammatory drug compound by weight.

11. The composition of claim 2, which contains about 0.0001% to about 99% of said non-steroidal anti-inflammatory drug (NSAID) compound by weight.

12. The composition of claim 11, which contains about 0.0001% to about 50% of said NSAID compound by weight.

13. The composition of claim 11, which contains about 0.001% to about 10% of said NSAID compound by weight.

14. The composition of claim 11, which contains about 0.01% to about 5% of said NSAID compound by weight.

15. The composition of claim 11, which contains about 0.1% to about 3% of said NSAID compound by weight.

16. The composition of claim 11, which contains about 0.5% to about 2% of said NSAID compound by weight.

17. The composition of claim 11, which contains about 1% of said NSAID compound by weight.

18. The composition of claim 1, wherein said botanical medicinal agent is selected from the group consisting of Achillea millefolium, Actaea racemosa, Allium sativum, Anethum graveolens, Amorphophallus konjac, Aquilare agollocha, Arnica spp., Aristolochia rotunda, Artemisia annua, Artemisia absinthium, Arum maculatum, Asimina spp., Astralagus membranaceus, Citrus aurantium, Crataegus spp., Cymbopogon spp., Cynara cardunculus, Digitalis lanata, Echinacea purpurea, Glycyrrhiza glabra, Hydratis canadensis, Hypericum perforatum, Lentinula edodes, Magnolia spp., Marrubium vulgare, Matricaria recruita, Melaleuca spp., Mentha piperita, Nepeta cataria, Nigella sativa, Oregano vulgare, Passiflora spp., Petasites spp., Phytolacca spp., Plantago spp., Poppiocious seediousphylla, Punica granatum, Rosa canina, Rubus fruticosis, Salvia lavandulaefolia, Salvia stenophylla, Sambocus spp., Serenoa repens, Silybum spp., Symphytum officinale, Tanacetum parthenium, Taracetum officinale, Tilia spp., Urtica dioica, Vaccinium spp., Valeriana officinalis, Vanilla spp., Verbascum thapsus, and Zingiber officinalis.

19. The composition of claim 18, wherein said botanical medicinal agent is Calendula.

20. The composition of claim 19, wherein said Calendula is Calendula officinalis.

21. The composition of claim 18, which contains about 0.0001% to about 99% of said Calendula by weight.

22. The composition of claim 18, which contains about 0.0001% to about 50% of said Calendula by weight.

23. The composition of claim 18, which contains about 0.001% to about 10% of said Calendula by weight.

24. The composition of claim 18, which contains about 0.01% to about 5% of said Calendula by weight.

25. The composition of claim 18, which contains about 0.1% to about 3% of said Calendula by weight.

26. The composition of claim 18, which contains about 0.5% to about 2% of said Calendula by weight.

27. The composition of claim 1, wherein said antihistamine is selected from the group consisting of fexofenadine, loratadine, desloratadine, azelastine, cetirizine and levocetirizine.

28. The composition of claim 2, wherein said NSAID compound is ibuprofen.

29. The method of treating dermatoses in a human in need thereof, which comprises topically applying to the skin of said human a topical composition of claim 1.

30. The method of treating swelling, redness, darkness or inflammation of the skin of a human in need thereof, which comprises topically applying to said skin a topical composition of claim 2.

31. The method of claim 29, wherein said method treats swelling of the skin under or around the eye of a human in need thereof.

32. The method of claim 29, wherein said method treats rosacea in a human in need thereof.

33. The method of claim 28, wherein said skin is on the face.

34. The method of claim 29, wherein said skin is on the face.

35. The method of claim 33, wherein said skin is the eye area.

36. The method of claim 34, wherein said skin is the eye area.
